(19) United States
(12) Patent Application Publication
(10) Pub. No.: US 2024/0358386 A1
(43) Pub. Date: Oct. 31, 2024

(54) THROMBECTOMY SYSTEM

(71) Applicant: Surmodics, Inc., Eden Prairie, MN (US)

(72) Inventors: Derek Stratton, Bloomigton, MN (US); Peter Bo, Eden Prairie, MN (US); Jordan Berg, Eden Prairie, MN (US); Alex Marine, Eden Prairie, MN (US)

(21) Appl. No.: 18/643,329

(22) Filed: Apr. 23, 2024

Related U.S. Application Data
(60) Provisional application No. 63/462,173, filed on Apr. 26, 2023.

(51) Int. Cl. A61B 17/221; A61B 17/32; A61B 17/3207; A61M 25/00

(52) U.S. Cl. CPC A61B 17/221; A61B 17/320725; A61B 2017/2215; A61B 2017/320012; A61M 2025/0004; A61M 25/0082

(57) ABSTRACT

A thrombectomy system includes an inner catheter, an outer catheter, an adjustable basket, and at least one debulking element. The outer catheter defines an outer catheter lumen. The inner catheter is slidably received within the outer catheter lumen such that the inner catheter is slidable relative to the outer catheter. The adjustable basket has a fenestrated structure extending between a proximal collar and a distal collar. The proximal collar is attached to a distal end of the outer catheter and the distal collar is attached to a distal end of the inner catheter. The at least one debulking element is positioned on the outer catheter proximal to the adjustable basket. The fenestrated structure is radially expanded by sliding the inner catheter in a first direction relative to the outer catheter and is radially collapsed by sliding the inner catheter in a second direction opposite the first direction.

### CLAIM OF PRIORITY

This patent application claims the benefit of priority, under 35 U.S.C. Section 119(e), to Stratton, et al., U.S. Provisional Patent Application Ser. No. 63/462,173, entitled “Thrombectomy System,” filed on Apr. 26, 2023 (Attorney Docket No. 3028.074PRV), which is hereby incorporated by reference herein in its entirety.

### TECHNICAL FIELD

This document pertains generally, but not by way of limitation, to an intravascular system, and more particularly, is for an intravascular thrombectomy system for removal of clot material.

### BACKGROUND

Thrombectomy procedures involve removal of clot material from an occluded (including partially occluded) blood vessel through open surgery or a catheter-based procedure. In open surgery, incisions are made to expose the occluded vessel and open the occluded vessel to expose the clot for direct removal of the clot material from the vessel. Open surgery is generally reserved for emergency situations or when a catheter-based procedure cannot be used. In contrast, catheter-based procedures involve navigating a catheter or sheath through the vasculature from an access site to the occlusion site and pulling clot material into the catheter or sheath for extraction from the body. The clot material is optionally pulled into the sheath by suction, mechanically snaring the clot material with a separate device, or a combination of both techniques.

Thrombectomy sheaths have profiles complementary to vasculature to navigate the vasculature and position the distal end of the sheath near the occlusion. As clot material accumulates in narrow regions of the vasculature, the distal end of the sheath may be narrow to access these narrower vessels.

### SUMMARY OF THE INVENTION

The present inventors have recognized, among other things, that a problem to be solved can include mobilizing large volume or mixed morphology clots for withdrawal through a sheath. The thin, elongated shape of the sheath requires a thicker or reinforced sheath wall to allow surgeons to maintain control of the distal tip of the inserted sheath, which further limits the available cross-sectional area through which the clot material can pass. Clot size and morphology itself can hinder catheter-based thrombectomy. Clots typically include insoluble material, such as fibrin, that becomes wedged within the vessel or adhered to the vessel wall or other trapped material. During extraction, clot material or the clot engagement device itself can plug the distal opening of the sheath or otherwise make it unable to be pulled back into the sheath. Over time, additional material becomes trapped behind the initial blockage increasing the overall volume of the clot. If the clot volume becomes too large, the sheath may become clogged or overwhelmed causing clot fragments to escape into the vasculature and embolize, which can cause additional health risks. If the sheath or the clot engagement device becomes plugged, open surgery may be required to remove a plugged sheath or an irretrievable clot engagement device that could not be safely retrieved.

Moreover, the older (“chronic”) clot material can naturally become denser as more solid material becomes entrapped within the clot material. Additional material that develops behind the chronic clot material can compress the chronic material and compress the overall clot into a dense mass of material. These dense plugs of clot material can be difficult to extract without maceration or fragmentation of the material. With mechanical thrombectomy, a clot engagement device is typically deployed distal to the clot material and pulled proximally toward a sheath mouth positioned proximate to the clot material. The pulling motion can compress the thinner, newer (“acute”) clot material trapped behind the already dense chronic clot material into a denser mass of material increasing the size of the naturally formed clot material plug. Aspiration thrombectomy gradually suctions portions of the clot material into the sheath mouth and avoids mechanical compression caused by mechanical thrombectomy. However, the suction generated during aspiration thrombectomy often cannot disrupt or macerate the denser plugs of thrombectomy. Likewise, wall adherent clot may be difficult to remove by suction-alone and may require mechanical disruption to scrape or otherwise separate the clot material from the vessel wall.

In an example, the present subject matter provides a solution to this problem with a basket catheter assembly having an adjustable (e.g., coring) basket and a debulking element (e.g., debulking basket). The adjustable or coring basket is positioned distal to the one or more debulking baskets and separated from the debulking baskets, for instance with a specified spacing. The basket catheter assembly, including the sheath and catheter, is advances across the clot material, and then the sheath is withdrawn to reveal the coring basket, which is positioned distal to the clot material and the debulking basket within the clot material.

The distal positioned coring basket is expanded to provide a clot engagement element, optionally distal to the clot. The one or more debulking baskets are expanded within the clot material to penetrate the clot material and optionally anchor the clot material within the baskets. The basket catheter assembly is withdrawn to engage the clot material with the coring basket and the debulking basket and mobilize the clot material toward the sheath.

The one or more debulking baskets are optionally expanded within the clot material to separately mobilize portions of the clot material proximal to the coring basket. This distributed mobilization effectively reduces the quantity of clot material mobilized by the trailing coring basket. In at least one embodiment, the basket catheter assembly includes a plurality of debulking baskets positioned proximal to the coring basket to anchor portions of the clot material and permit the distributed mobilization of the clot material by one or more of the debulking baskets and the coring basket.

In at least one example of the present subject matter, the basket catheter assembly includes an adjustable coring basket and one or more debulking baskets. The coring basket includes a fenestrated structure having a tapered distal end and an open proximally facing mouth. At least two basket arms project proximally from opposing edge positions of the open proximal mouth of the coring basket before tapering to a connecting element, that links the two arms. The coring basket is withdrawn proximally by the basket catheter assembly to collect clot material through the open mouth. In another example, the connecting element (coupled with the basket arms) is moved distally to radially expand the coring element. For instance, the two basket arms spread the proximal mouth as the connecting element is moved distally. In at least one embodiment, the fenestrated structure of the coring basket includes notches along the mouth edge between the arms. The notches are expandable by the arms as the connecting element is moved distally to further enlarge the proximal mouth and permit one or more of reception of larger clot or complementary fitting along larger vessel walls. The connecting element is conversely moved proximally to radially collapse the fenestrated structure and the open mouth.

The basket catheter assembly includes an inner shaft slidably received within the outer shaft. A distal end of the coring basket is attached to the inner shaft and a connecting element of the coring basket is attached to the outer shaft. The inner shaft can be slid within the outer shaft in a first direction to draw the distal end and the connecting element together to radially expand the coring basket and enlarge the proximal mouth. This enlarged proximal mouth is able to collect clot material and prevent the clot material from sliding past the coring basket. Likewise, the inner shaft can be slid relative to the outer shaft in a second direction opposite the first direction to separate the distal end from the connecting element further apart to radially collapse the coring basket. The collapsing of the coring basket allows the coring basket to be re-sheathed more easily. This more effectively captures the clot material for extraction upon collapsing and removal of the sheath, with the coring basket and clot material within the sheath. In this arrangement, the inner shaft can be moved relative to the outer shaft to radially expand the coring basket and enlarge the proximal facing mouth (for collection of the clot material) or collapse the coring basket and the proximal facing mouth (for extraction of the clot material).

In at least one example of the present subject matter, the basket catheter assembly includes a handle assembly for manipulating the basket catheter assembly and selectively moving the inner shaft relative to the outer shaft. A proximal end of the outer shaft can be fixedly connected to the handle assembly while a proximal end of the inner shaft is slidably received within the handle assembly. The handle assembly includes a control knob rotatable to move the inner shaft axially relative to the outer shaft. In an embodiment, the proximal end of the inner shaft includes a lead screw that is optionally engaged by the rotatable element to translate the rotation of the rotatable element into axial movement of the inner shaft relative to the outer shaft. Generally, the control knob is rotated in a first direction to advance the inner shaft distally relative to the outer shaft to radially collapse the coring basket. Similarly, the rotatable element is rotated in a second direction, opposite the first direction, to withdraw the inner shaft proximally relative to the outer shaft to radially expand the coring basket. The threaded arrangement permits moving the inner shaft to a predetermined or specified position relative to the outer diameter to expand and maintain the coring basket at a predetermined or specified radial diameter during withdrawal of the basket catheter assembly toward the procedural sheath.

In an embodiment, the handle assembly includes a slider for manually advancing the inner shaft distally apart from the control knob. The slider disengages the inner catheter from the lead screw to advance the inner catheter distally, independently of the lead screw, and to radially collapse the coring basket. This allows the operator to quickly collapse the coring basket if a narrow portion of the vessel is encountered or the clot material burden may overcome the coring basket. The handle assembly includes a return spring that opposes the slider to re-engage the inner catheter to the lead screw when the slider is released. Generally, the operator depresses the slider to temporarily collapse the coring basket before releasing the slider to permit the coring basket to re-expand to the prior radial diameter.

### BRIEF DESCRIPTION OF THE FIGURES

FIG. 1 is a schematic sideview of a thrombectomy system according to an embodiment of the present invention deployed within a vessel.

FIG. 2 is a side view of a thrombectomy system according to an embodiment of the present invention.

FIG. 3 is a top view of the thrombectomy system depicted in FIG. 2.

FIG. 4 is a partial side view of a basket catheter assembly according to an embodiment of the present invention.

FIG. 5 is a partial front view of a coring basket according to an embodiment of the present invention.

FIG. 6 is a partial side view of a coring basket according to an embodiment of the present invention.

FIG. 7A is a side view of a thrombectomy system according to an embodiment of the present invention with a delivery sheath in a fully sheathed position.

FIG. 7B is a side view of the thrombectomy system depicted in FIG. 7A with the delivery sheath positioned to unsheathe a coring basket.

FIG. 7C is a side view of the thrombectomy system depicted in FIG. 7A with the delivery sheath positioned to unsheathe the coring basket and a first debulking basket.

FIG. 7D is a side view of the thrombectomy system depicted in FIG. 7A with the delivery sheath positioned to unsheathe the coring basket, the first debulking basket, and a second debulking basket.

FIG. 8 is a partial cross-sectional side view of a thrombectomy system according to an embodiment of the present invention.

FIG. 9A is a partial side view of a thrombectomy system according to an embodiment of the present invention with a coring basket in an expanded configuration.

FIG. 9B is a cross-sectional side view of a handle assembly of a thrombectomy system according to an embodiment of the present invention with a translation assembly position to expand the coring basket.

FIG. 10A is a partial side view of the thrombectomy system depicted in FIG. 9A with the coring basket in a collapsed configuration.

FIG. 10B is a cross-sectional side view of the handle assembly depicted in FIG. 9B with the translation assembly positioned to collapse the coring basket.

FIG. 11A is a partial side view of the thrombectomy system depicted in FIG. 9A with the coring basket in a partially collapsed configuration.

FIG. 11B is a partial side view of the thrombectomy system depicted in FIG. 9B with a knockdown assembly positioned to partially collapse the coring basket.

FIG. 12 is a side view of a thrombectomy system according to an embodiment of the present invention.

FIG. 13 is a partial side view of the thrombectomy system depicted in FIG. 12.

FIG. 14 is a cross-sectional side view of the thrombectomy system depicted in FIG. 12.

FIG. 15 is a side view of a thrombectomy system according to an embodiment of the present invention.

FIG. 16 is a partial side view of the thrombectomy system depicted in FIG. 15.

FIG. 17 is a cross-sectional side view of the thrombectomy system depicted in FIG. 15.

FIG. 18 is a cross-sectional side view of the thrombectomy system depicted in FIG. 15.

### DETAILED DESCRIPTION OF THE FIGURES

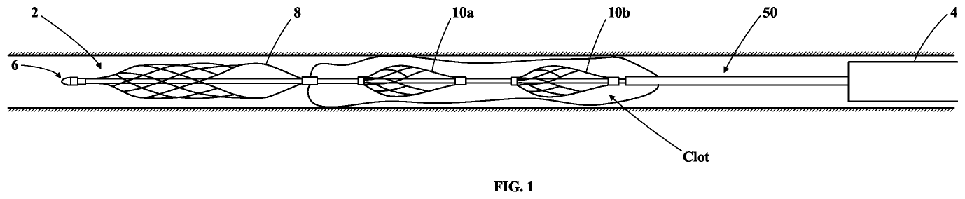

As shown in FIG. 1, a thrombectomy system 2 for removing clot material from a blood vessel, according to an embodiment of the present invention, includes a procedural sheath 4 and a basket catheter assembly 6 having an adjustable (e.g., coring) basket 8 and at least one debulking element (e.g., basket) 10 (10a, 10b) for engaging and mobilizing clot material. Generally, the procedural sheath 4 can be advanced through the vasculature from an access site to position the distal end of the procedural sheath proximate to the clot material. The basket catheter assembly 6 is then advanced through the clot material to position at least the coring basket 8 distal to the clot material. Each debulking basket 10 is positioned on the basket catheter assembly 6 at a position proximal to the coring basket 8. As illustrated in FIG. 1, the positioning of the coring basket 8 and each debulking basket 10 allows the coring basket 8 to be deployed distally to the clot material and each debulking basket 10 to be deployed within the clot material. As needed, the operator can also further advance the basket catheter assembly 6 such that the coring basket 8 and one or more debulking basket 10 is positioned distal to the clot material. After deployment of the coring basket 8 and each debulking basket 10, the basket catheter assembly 6 can be withdrawn toward the procedural sheath 4 to mobilize the clot material towards and eventually into the procedural sheath 4 for removal from the body. The coring basket 8 and each debulking basket 10 are attached to the basket catheter assembly 6 such that withdrawing the basket catheter assembly 6 moves the coring basket 8 and each debulking basket 10 together. When deployed within or near the clot material, the proximal positioning of the debulking baskets 10 causes each debulking basket 10 to separately mobilize a portion of the clot material prior to trailing the coring basket 8. This arrangement partitions or divides the volume of clot material between the coring basket 8 and the debulking baskets 10, reducing the burden on each basket and decreasing the likelihood that clot material overwhelms or escapes capture. Further, as pulling the coring basket 8 or the debulking baskets 10 proximally through the clot material will cause loosely packed clot material to compress against the basket, forming a denser mass of material that can plug the procedural sheath 4, dividing the clot material between the debulking baskets 10 minimizes the size of any dense plugs that can form and reduces the likelihood of the procedural sheath 4 becoming plugged. Similarly, the engagement of or movement of the proximally positioned debulking baskets 10 through the clot material can cause some of the clot material to fragment into smaller pieces. The smaller fragments can be easier to collect within the trailing coring basket 8.

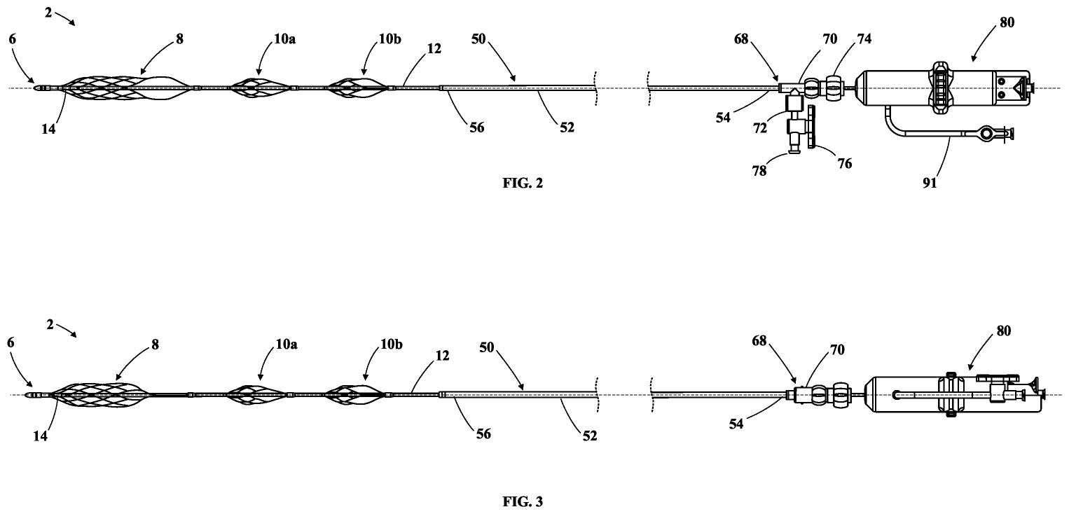

As illustrated in FIGS. 2-3, in at least one embodiment, the basket catheter assembly 6 includes at least a first debulking basket 10a and a second debulking basket 10b. Additional debulking baskets 10a, 10b can further partition and separately mobilize portions of the clot material. Likewise, fewer debulking baskets 10a, 10b can be provided to shorten the effective length of the basket catheter assembly 6. Depending on the position of vasculature in which the clot has formed, the available length of vessel where the thrombectomy system 2 can be deployed distal to the clot material may be limited. For example, the clot may form in a region of the vasculature where only a limited length of vessel wide enough to receive the thrombectomy system 2 may be available or a sensitive portion of the vasculature or organ is immediately distal to the clot.

As shown in FIG. 2-4, the basket catheter assembly 6 includes an outer catheter 12 and an inner catheter 14 slidably received within the outer catheter 12. The coring basket 8 is attached to a distal portion of the outer catheter 12 and a distal portion of the inner catheter 14, wherein sliding the inner catheter 14 relative to the outer catheter 12 expands and contracts the coring basket 8. The debulking elements (e.g., baskets) 10a, 10b are positioned on the outer catheter 12 proximal to the coring basket 8 at a predetermined distance. In at least one embodiment, the distance from the proximal end of the coring basket 8 to the proximal end of the first debulking basket 10a can be about 40 to 100 mm. In yet another embodiment, the distance can be about 70 mm. The debulking baskets 10a, 10b are spaced apart at a predetermined distance. In at least one embodiment, the distance from the proximal end of a first debulking basket 10a to a proximal end of a second debulking basket 10b can be about 40 to 100 mm. In yet another embodiment, the distance can be about 70 mm. The spacing between the coring basket 8 and the debulking baskets 10a, 10b can correspond to the specific vessel to be targeted and the typical size and volume of the clot found therein. The spacing between the proximal ends of the of the coring basket 8 and the debulking basket 10a, 10b can be about the same to maintain even spacing between the baskets. In at least one embodiment, the inner catheter 14 defines an inner lumen 15 extending through the inner catheter 14 at least for receiving a procedural guidewire (not shown) for delivering the thrombectomy system 2 to the clot. The inner lumen 15 can extend from the distal end to the proximal end of the inner catheter 14 for over-the-wire delivery of the thrombectomy system 2. Alternatively, the inner lumen 15 can extend from the distal end to an exit opening distal to the proximal end of the inner catheter 14 for rapid-exchange delivery of the thrombectomy system 2.

As shown in FIG. 4, the coring basket 8 includes a basket wall 16 having a closed distal end 18 and an open proximal end 20. In an embodiment, the basket wall 16 can be expanded radially to adopt a generally cylindrical shape having a tapered distal end 18 and an open proximal mouth 20. When expanded, the basket wall 16 and closed distal end 18 define an internal volume for receiving clot material. Generally, the expanded coring basket 8 can be withdrawn proximally through the clot material as the basket catheter assembly 4 is withdrawn toward the procedural sheath, which collects clot material in the mouth of the coring basket 6 for collection within the internal volume. The basket wall 16 can be elongated axially to increase the internal volume for receiving clot. In some examples, the coring basket 8 may be up to 15 mm in diameter. In some examples, the coring basket 8 may be within a range of 2-4 mm in diameter. In some examples, the coring basket 8 may be within a range of 3.5-6 mm in diameter. In some examples, the coring basket 8 may be within a range of 6-10 mm in diameter. As the coring basket 8 trails the debulking baskets 10a, 10b when the basket catheter assembly 4 is withdrawn toward the procedural sheath, the coring basket 8 can require additional capacity for collecting excess clot material not collected by the debulking baskets 10a, 10b or clot fragments macerated by the debulking basket 10a, 10b. In at least one embodiment, the internal volume of the basket wall 10 can be greater than the internal volume of each debulking basket 10a, 10b. The structure of the coring basket 8 can further increase the carrying capacity of the coring basket 8. As illustrated in FIGS. 4-5, in at least one embodiment, the basket wall 10 includes a fenestrated structure formed by a cut hypotube comprising stainless steel, cobalt chromium, nitinol, or other resilient material. Alternatively, the basket wall 16 includes a woven structure formed by weaving wires comprising stainless, steel, cobalt chromium, polymer, or other resilient material. The struts of the fenestrated structure or the wires of the woven structure can snare or engage clot material entering the internal volume or passing along the exterior of the coring basket 8 as the coring basket 8 is pulled through the clot material. The basket wall 16 can be elongated to increase the internal volume available to receive clot material as well as the surface area of the basket wall 10 available to snare or engage the clot material. The struts or wires forming the lip of the proximal mouth 20 can be thickened, oriented, or reinforced to resist deformation of the coring basket 8 at the proximal mouth 20 as the edges of the proximal mouth 20 engage clot material. The reinforcement allows the edges of the proximal mouth 20 to passed close to or scraped along the surface of the vessel to separate or scrape wall adherent clot material from the vessel wall for capture within the coring basket 6 or snared by the struts or wires of the basket wall 16.

As shown in FIG. 4, the coring basket 8 optionally includes at least two basket arms 22a, 22b that project proximally from opposite positions along the edge of the proximal mouth 20. The basket arms 22a, 22b taper radially inward as the basket arms 22a, 22b extend proximal from the edge of the proximal mouth 20. The basket arms 22a, 22b are connected at the proximal end of the basket arms 22a, 22b by a proximal collar 24. The proximal collar 24 is attached to a distal portion of the outer catheter 12. The distal end 18 includes a distal collar 26 attached to a distal portion of the inner catheter 14. Generally, the inner catheter 14 can be withdrawn proximally relative to the outer catheter 12 thereby reducing the distance between the proximal collar 24 and the distal collar 26, which compresses the basket wall 16 and radially expands the coring basket 8. As the inner catheter 14 is initially withdrawn, the opposing positions of the basket arms 22a, 22b press against opposite points on the proximal mouth 20 elongating and enlarging the proximal mouth 20. In at least one embodiment, the proximal mouth 20 can define at least one notch portion 28, which opens as the basket arms 22a, 22b elongate the proximal mouth 20 to increase the cross-sectional area of the enlarged proximal mouth 20. Similarly, the inner catheter 14 can be advanced distally relative to the outer catheter 12 thereby increasing the distance between the proximal collar 24 and the distal collar 26, which elongates the basket wall 16 and radially collapses the coring basket 8. The inner catheter 14 can be positioned at a plurality of positions relative to the outer catheter 12 to expand or contract the coring basket 8 to a desired radial diameter such as the vessel diameter. This arrangement allows the edge of the proximal mouth 20 to be positioned approximate to the vessel wall for scraping or separating wall adherent clot from the vessel wall for collection within the inner volume of the coring basket 8. As the inner catheter 14 is withdrawn proximally relative to the outer catheter 12 to compress the coring basket 8 and force radial expansion of the coring basket 8, the axial force required to move the inner catheter 14 further proximally increases as the coring basket 8 expands further radially. The inner catheter 14 can buckle if the coring basket 8 is too resistant to further compression of the coring basket 8. As shown in FIGS. 4 and 6, in at least one embodiment, the outer catheter 12 optionally includes a support section 30 that extends distally beyond the proximal collar 24 over a portion of the inner catheter 14 extending through the inner volume of the coring basket 8. The support section 28 reinforces the inner catheter 14 where the risk of deformation is greatest when the inner catheter 14 is withdrawn proximally to radially expanding the coring basket 8 to reduce the likelihood of buckling of the inner catheter 14. The distal end of the support section 28 can also abut the proximal collar 26 preventing further proximal withdrawal of the inner catheter 14. This arrangement provides a stop preventing further radial expansion of the coring basket 8, which can cause buckling of the inner catheter 14 or eversion of the coring basket 8.

As shown in FIG. 4, in an embodiment, each debulking basket 10a, 10b includes a basket wall 32 having a closed distal end 34 and an open proximal end 36. In an embodiment, the basket wall 32 can be expanded radially to adopt a generally cylindrical shape having a tapered distal end 34 and an open proximal mouth 36. When expanded, the basket wall 32 and closed distal end 34 define an internal volume for receiving clot material. The generally cylindrical shape can provide a longer internal volume for receiving clot within each debulking basket 10a, 10b. In another embodiment, the basket wall 32 can adopt a spherical, Toric, disc, or other shape with a shorter axial length when radially expanded. The shorter axial length reduces the available internal volume for receiving clot material but eases the penetration of the clot material by the basket wall 32 as the debulking basket 10a, 10b is expanded radially. This arrangement allows each debulking basket 10a, 10b to penetrate deeper into the clot material when expanded within the clot material, which improves partitioning of the clot material by the debulking baskets 10a, 10b. As with the coring basket 6, the structure of the debulking baskets 10a, 10b can increase the carrying capacity of the debulking baskets 10a, 10b. As illustrated in FIG. 4, in at least one embodiment, the basket wall 32 includes a fenestrated structure formed by a cut hypotube comprising stainless steel, cobalt chromium, nitinol, or other resilient material. Alternatively, the basket wall 32 optionally includes a woven structure formed by weaving wires comprising stainless, steel, cobalt chromium, polymer, or other resilient material. The struts of the fenestrated structure or the wires of the woven structure can snare or engage clot material entering the internal volume or passing along the exterior of the debulking baskets 10a, 10b as the debulking baskets 10a, 10b are pulled through the clot material. The basket wall 32 can be elongated to increase the internal volume available to receive clot material as well as the surface area of the basket wall 32 available to snare or engage the clot material. The struts or wires forming the lip of the proximal mouth 36 can be thickened or reinforced to resist deformation of the debulking baskets 10a, 10b at the proximal mouth 36 as the edges of the proximal mouth 36 engage clot material. The reinforcement allows the edges of the proximal mouth 36 to passed close to or scraped along the surface of the vessel to separate or scrape wall adherent clot material from the vessel wall for capture within the debulking baskets 10a, 10b or snared by the struts or wires of the basket wall 32. In an embodiment, the struts or wires forming the lip of the basket wall 32 can be reinforced, thickened, or more densely woven at the radially outermost portion. This arrangement resists deformation of the debulking baskets 10a, 10b at the radially outermost portion creating a cutting portion for better penetration of the clot material by the debulking baskets 10a, 10b as the debulking baskets 10a, 10b expands radially.

As shown in FIG. 4, in an embodiment, each debulking basket 10a, 10b optionally includes a plurality of basket arms 38 that project proximally from the edge of the proximal mouth 36. The basket arms 38 taper radially inward as the basket arms 38 extend proximally from the edge of the proximal mouth 36. Each debulking basket 10a, 10b includes a fixed collar 40 attached to a position on the outer catheter 12, wherein the proximal end of each basket arm 38 is connected to the fixed collar 40. Each debulking basket 10a, 10b further includes a sliding collar 42 slidably received on the outer catheter 12, wherein the distal end 34 of the debulking basket 10a, 10b is connected to the sliding collar 42. The sliding collar 36 is moveable axially along the outer catheter 12 relative to fixed collar 40 between an expanded configuration and a collapsed configuration. Generally, in the collapsed configuration, the sliding collar 42 is moved axially apart from the fixed collar 40 to pull the basket arms 28 and the basket wall 32 radially inward to collapse the debulking basket 10a, 10b. Similarly, in the expanded configuration, the sliding collar 42 is moved toward the fixed collar 40 pushing the basket arms 38 and the basket wall 32 radially outward to expand the debulking basket 10a, 10b. The basket wall 32 and basket arms 32 includes resilient materials and be shaped such to be biased toward the expanded configuration. This arrangement allows the debulking basket 10a, 10b to self-expand when uncovered or unsheathed.

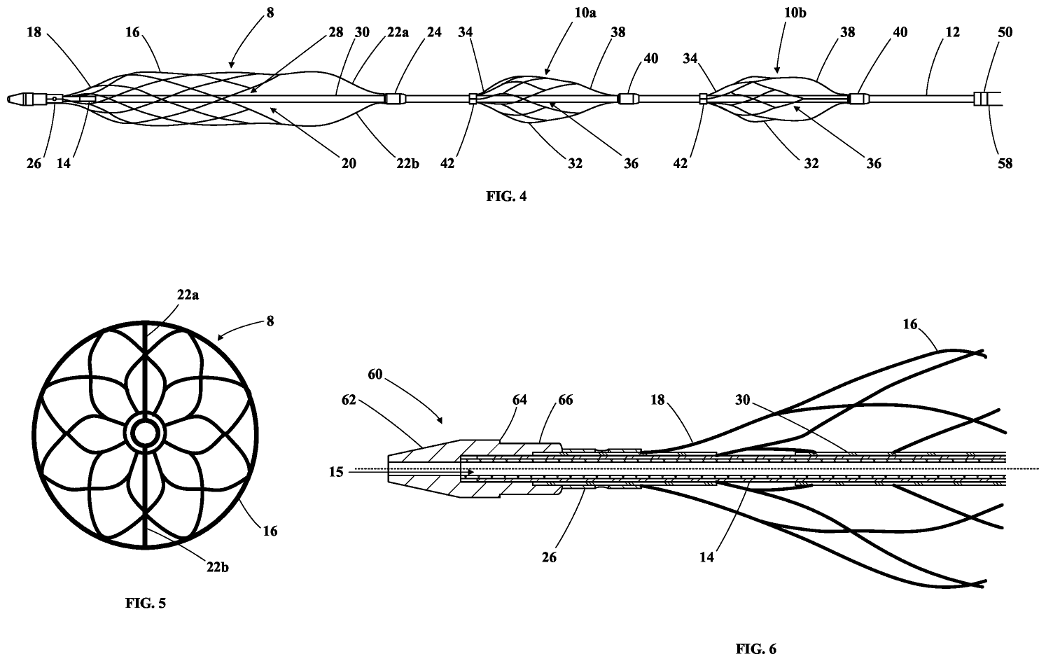

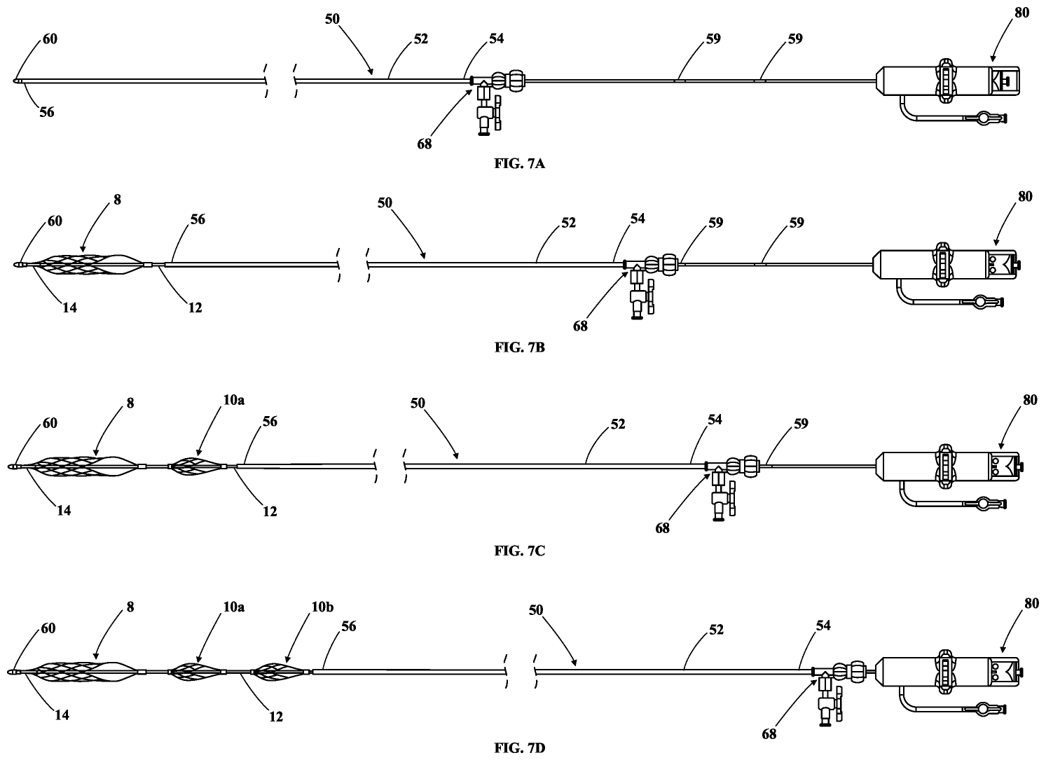

As shown in FIGS. 1-3 and 7A-D, the thrombectomy system 2 includes a delivery sheath 50 slidable over the basket catheter assembly 6. The delivery sheath 50 includes an elongated sheath body 52 having a proximal end 54 and a distal end 56 and defining a lumen extending between the proximal end 54 and the distal end 56. The basket catheter assembly 6 is slidably receivable within the lumen of the delivery sheath 50 wherein the delivery sheath 50 is slidable axially over the basket catheter assembly to sheath and unsheathe the coring basket 8 and the debulking baskets 10a, 10b. The distal mouth 56 of the sheath body 52 can be reinforced to prevent distortion of the distal mouth 56 as the distal end 56 is slid over the coring basket 8 and the debulking baskets 10a, 10b. In at least one embodiment, the delivery sheath 50 optionally includes a radiopaque band 58 positioned at the distal end 56 for identifying the position of the distal end 56 with fluoroscopy or other imaging techniques during a surgical procedure. As illustrated in FIGS. 7A-D, the delivery sheath 50 can be advanced distally over the basket catheter assembly 6 to fully sheath the coring basket 8 and the debulking baskets 10a, 10b for delivery of the thrombectomy system 2 to and across the clot. Following delivery and positioning of the basket catheter assembly 6 and the delivery sheath 50, the delivery sheath 50 can be withdrawn proximally a first predetermined distance to unsheathe the coring basket 8. The unsheathed coring basket 8 can then expand radially to at least partially occlude the vessel by self-expansion, manual expansion by the movement of the inner catheter 14 as described above, or combination thereof. The delivery sheath 50 can be withdrawn further proximally to unsheathe one or more debulking baskets 10a, 10b, which can then self-expand radially to at least partially occlude the vessel. As illustrated in FIGS. 7A-D, in at least one embodiment, the outer catheter 12 optionally includes at least one marker band 59 denoting which baskets have been unsheathed and against which the proximal end 54 of the elongated sheath body 50 can be aligned to unsheathe the desired combination of baskets. The spacing of the baskets and the movement of the delivery sheath 50 allows an operator to select the number of baskets to be expanded for treatment of clot. As discussed above, depending on the location the useable vessel length distal to the clot may be limited. Similarly, for shorter clots or smaller volumes, an operator may choose to use fewer baskets to minimize trauma to the vessel wall.

As shown in FIG. 6, the basket catheter assembly 6 optionally includes a distal tip 60 having a tapered distal face 62 and a proximal-facing shoulder portion 64 against which the distal end 56 of the delivery sheath 50 can abut when the delivery sheath 50 fully sheaths the coring basket 8 and the debulking baskets 10a, 10b. In at least one example, the distal tip 60 optionally includes an alignment surface 66 for engaging the distal mouth 56 and aligning the distal end 56 of the delivery sheath 50 with the distal tip 60 of the basket catheter assembly 6. When fully sheathed, the tapered distal face 62 of the distal tip 60 of the basket catheter assembly 6 provides an atraumatic dilator head for the delivery sheath 50.

As shown in FIG. 2, the delivery sheath 50 optionally includes a hub assembly 68 having a primary branch 70 and a side branch 72. The primary branch 70 is axially aligned with the elongated sheath body 52 and defines a lumen extending the lumen of the elongated sheath body 52. The primary branch 70 includes a locking element 74 such as a locking screw or a friction lock for engaging the outer catheter 12 of the basket catheter assembly 6. The locking element 74 can be tightened to fix the delivery sheath 50 at a particular position axially along the outer catheter 12. The side branch 72 defines a lumen fluidly connected to the lumen of the primary branch 70. The side branch 72 includes a valve 76 and a port 78. The port 78 can be used to deliver fluid for flushing the delivery sheath 50 or applying vacuum for aspirating the delivery sheath 50, while the valve 76 can control fluid flow through the side branch 72.

As shown in FIGS. 2-3 and 8-11B, in an embodiment, the thrombectomy system 2 includes a handle assembly 80 operably connected to the proximal end of the basket catheter assembly 6. The handle assembly 80 includes a handle body 82 and a translation assembly 84 for axially advancing and withdrawing the inner catheter 14 of the basket catheter assembly 6. Generally, the outer catheter 12 is fixedly attached to the handle body 82 while the translation assembly 84 is configured to advance and withdraw the inner catheter 14 relative to the outer catheter 12 to contract and expand the coring basket 8.

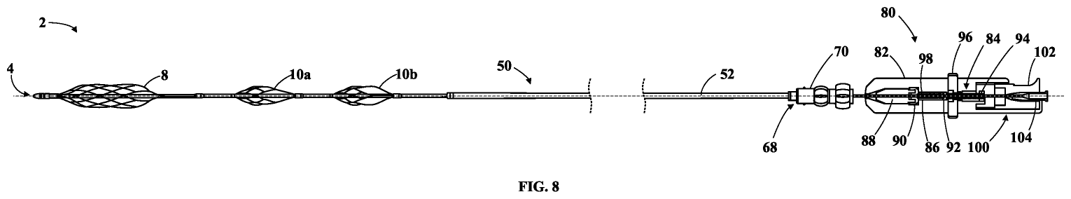

As shown in FIG. 8-11B, in an embodiment, the handle body 82 defines a translation lumen 86 for slidably receiving the inner catheter 14. The handle body 82 is configured to receive the proximal end of the outer catheter 12 and align the outer catheter 12 with the translation lumen 86. In at least one embodiment, the outer catheter 12 includes a proximal hub 88 having a hemostasis valve 90, wherein the handle body 82 is configured to receive the proximal hub 88 and align the hemostasis valve 90 with the translation lumen 86. In this configuration, the inner catheter 14 is slidably received within the translation lumen 86 and further slidably received within the outer catheter 12 within the hemostasis valve 90. The hemostasis valve 90 includes a seal body permitting translation of the inner catheter 14 relative to the outer catheter 12 while preventing leakage between the outer catheter 12 and the inner catheter 14. In at least one embodiment, the outer catheter 12 optionally includes a side arm 91 fluidly connected to the proximal hub 88 for flushing or aspirating the outer catheter 12.

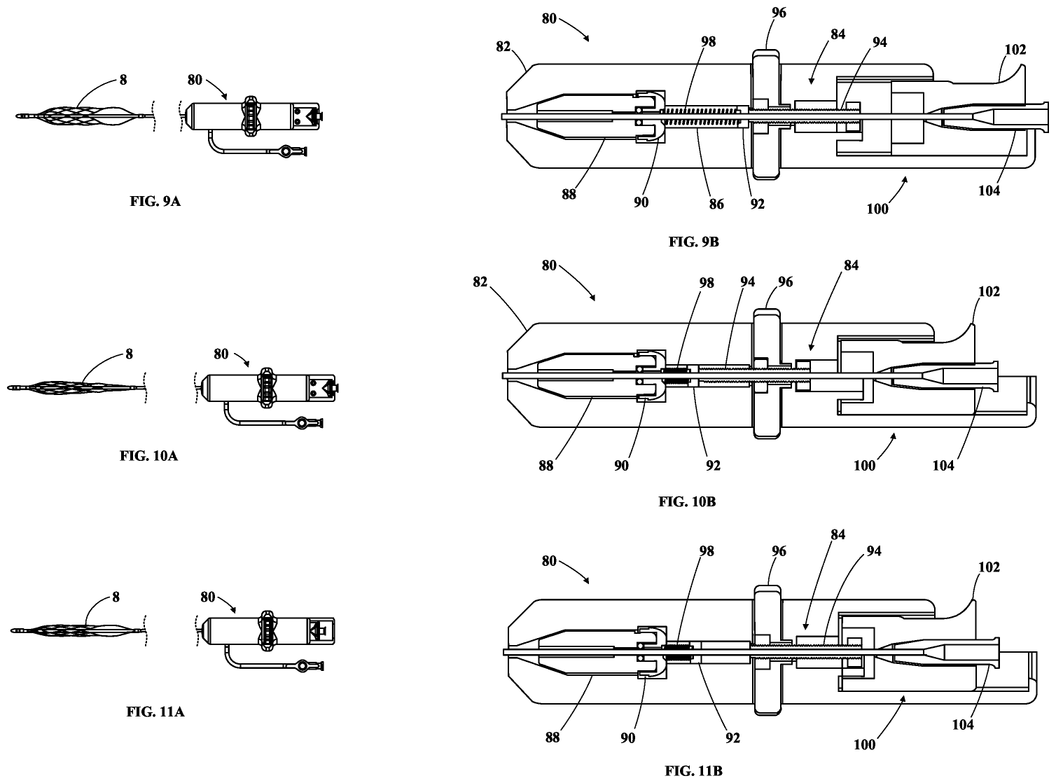

As shown in FIG. 8-11B, in an embodiment, the inner catheter 14 includes a land 92 projecting radially from a proximate portion of the inner catheter 14. The land 92 is slidably receivable within the translation lumen 86, wherein the translation lumen 86 guides axial translation of the land 92 and correspondingly the inner catheter 14 to maintain alignment of the inner catheter 14 with the outer catheter 12 during axial translation of the inner catheter 14. The translation assembly 84 includes a lead screw 94 positioned proximate to the land 92 within the translation lumen 86 and axially moveable within the translation lumen 86. The translation assembly 84 includes a rotatable element 96 operably engageable to the threads of the lead screw 94 wherein rotation of the rotatable element 96 is translated to axial movement of the lead screw 94 within the translation assembly 84. As illustrated in FIGS. 9A-10B, the rotatable element 96 is rotatable in a first direction to advance the lead screw 94 distally against the land 92 of the inner catheter 14 to push and correspondingly advance the inner catheter 14 distally. Similarly, the rotatable element 96 is rotatable in a second direction, opposite the first direction, to withdraw the lead screw 94 proximally. The translation assembly 84 includes a return spring 98 positioned distally to the land 92 within the translation lumen 86. In operation, rotating the rotatable element 96 in the first direction to advance the lead screw 94 and the land 92 of the inner catheter 14 distally compresses the return spring 98. When the rotatable element 96 is rotated in the second direction to withdraw the lead screw 94 proximally, the return spring 98 can expand pushing the land 92 of the inner catheter 14 proximally until the land 92 abuts against the lead screw 94. As discussed above, the axial translation of the inner catheter 14 relative to the outer catheter 12 expands and contracts the coring basket 8. In this manner, the translation assembly 84 is operable to translate the inner catheter 14 relative to the outer catheter 12 to expand or contract the coring basket 8.

As shown in FIGS. 8-11, in an embodiment, the handle assembly 82 includes a knockdown assembly 100 for manually collapsing or expanding the coring basket 8 radially. The blood vessel may have a varied diameter over the length of the vessel due to natural variation in the vessel diameter or wall adherent clot or plaque. A mechanical thrombectomy device with a fixed diameter or aggressively expanded clot engagement basket or element can injure the vessel wall or become trapped in wall adherent plaque or heavily adherent clot. The knockdown assembly 100 is configured to permit temporary disengagement of the coring basket 8 from the translation assembly 84 to radially collapse the coring basket 8 in response to narrowing vessel diameter or wall adherent plaque or clot material. The coring basket 8 can also be narrowed to reduce the cross-sectional area of the proximal mouth 20 of the coring basket 8, which reduces the amount of clot material collected as the coring basket 8 is pulled through the vessel and the likelihood that the coring basket will become trapped in the wall adherent material. The knockdown assembly 100 includes a slider 102 slidably mounted to the housing body 82 and configured to translate axially relatively to the housing body 82. The inner catheter 14 can extend proximally through the lead screw 94 to the slider 102. The proximal end of the inner catheter 14 can be fixedly attached to the slider 102, wherein axial translation of the slider 102 axially translates the inner catheter 14 to expand or collapse the coring basket 8. As illustrated in FIGS. 11A-B, the slider 102 can be advanced distally to advance the inner catheter 14 relative to the outer catheter 12 to elongate and radially collapse the coring basket 8. As the inner catheter 14 is advanced distally by the slider 102, the land 92 is advanced distally relative to the lead screw 94, which remains at the current position. Advancing the land 92 distally also compresses the return spring 98, which pushes the land 92 proximally when the slider 102 until the land 92 re-engages the lead screw 94 and re-expands the coring basket 8 to the radial diameter prior to actuation of the slider 102.

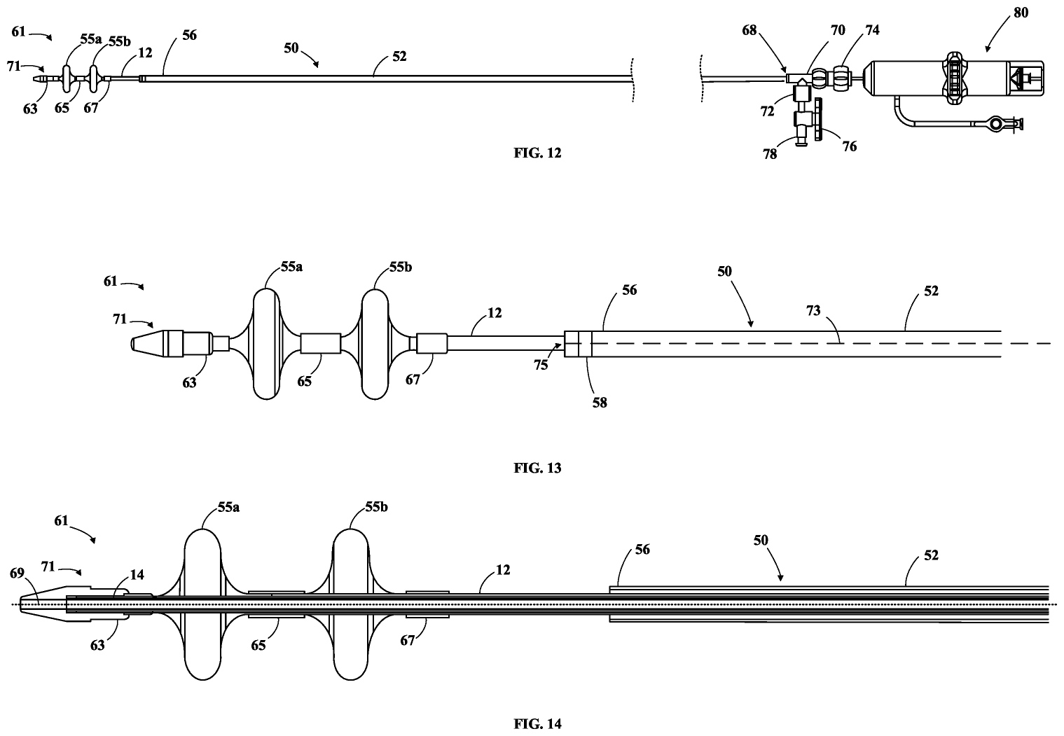

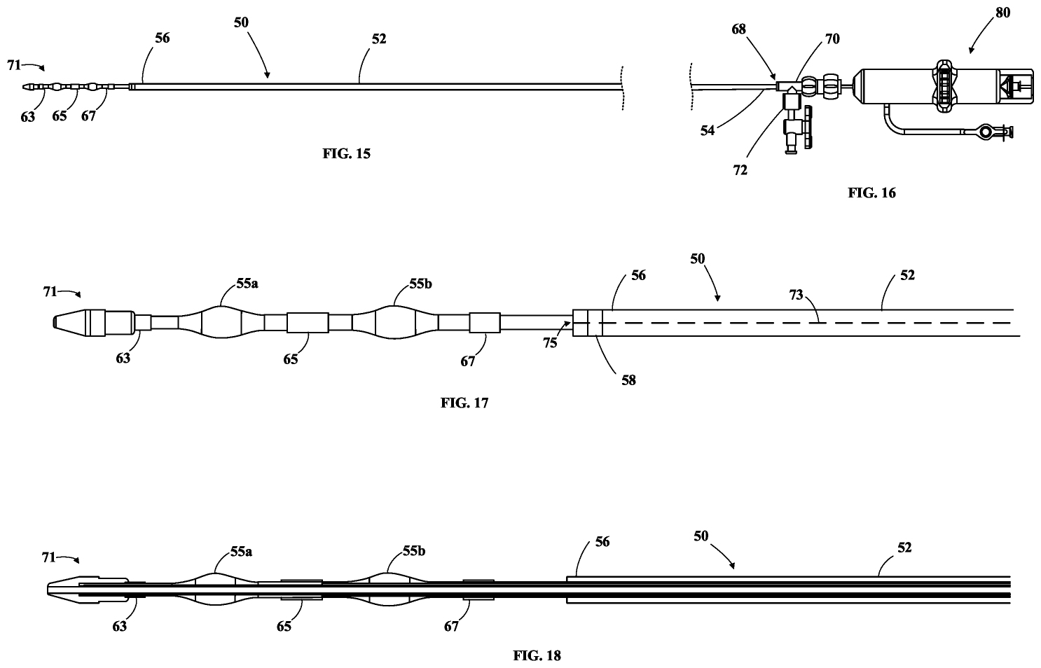

As shown in FIGS. 12-14, the thrombectomy system 61 includes an outer catheter 12 and an inner catheter 14 slidably received within the outer catheter 12. The thrombectomy system 61 includes at least two expandable brush elements 55a, 55b positioned along the outer catheter 12. The at least two expandable brush elements 55a, 55b are connected to a distal collar 63 and a proximal collar 67. The proximal collar 67 is attached to a distal portion of the outer catheter 12. The distal collar 63 is attached to a distal portion of the inner catheter 12. Generally, as shown in FIGS. 12-14, the inner catheter 14 can be withdrawn proximally relative to the outer catheter 12 thereby reducing the distance between the proximal collar 67 and the distal collar 63, which compresses and radially expands the thrombectomy system 61. And vice versa, as shown in FIGS. 15-18, as the inner catheter 14 is moved in a distal direction from the outer catheter 12, the distance between the proximal collar 67 and the distal collar 63 is increased, thereby decompressing and radially contracting the thrombectomy system 61.

Similar to what is shown in FIG. 7A, a coring basket can be sheathed in the delivery sheath 50 just proximal from the expandable brush element 55b. By moving the outer catheter 12 more distal or moving the delivery sheath more proximal, the coring basket is exposed (as is similarly shown in FIG. 7B, but without an expandable brush element shown distal to the coring basket). In some embodiments, at least one debulking element (e.g., basket) can be sheathed proximally to the expandable brush element 55b or proximal to a coring basket. The expandable brush elements 55a, 55b are positioned on the inner catheter 12 at a predetermined distance. Likewise, a coring basket or debulking elements can be at predetermined distances from each other or from the expandable brush elements 55a, 55b. The spacing between the expandable brush elements 55a, 55b, or an adjustable basket can correspond to the specific vessel to be targeted and the typical size and volume of the clot found therein. In at least one embodiment, the inner catheter 14 defines an inner lumen 69 extending through the inner catheter 14 at least for receiving a procedural guidewire (not shown) for delivering the thrombectomy system 61 to the clot. The inner lumen 15 can extend from the distal end to the proximal end of the inner catheter 14 for over-the-wire delivery of the thrombectomy system 61. Alternatively, the inner lumen 15 can extend from the distal end to an exit opening distal to the proximal end of the inner catheter 14 for rapid-exchange delivery of the thrombectomy system 61.

The expandable brush elements 55a, 55b focally abrade a clot or wall adherent or treat an area where there are any abrupt vessel diameter reductions. In some examples, the expandable brush elements 55a, 55b are mesh wire disks of specified diameter. The disk diameters can be modulated to adapt to vessel diameter in order to be under-sized, line-to-line, or have interference for increased or maximum vessel contact. In some examples, any number of mesh wire disks can be stacked along the inner catheter 14 and are used to de-clog a lumen when removing the thrombectomy system 61. The expandable brush elements 55a, 55b includes a woven mesh structure formed by weaving wires comprising stainless, steel, cobalt chromium, polymer, or other resilient material. The expandable brush elements can remove, snare, or engage clot material within the vessel as the expandable brush element 55a, 55b contacts or is pulled across the clot material. The expandable brush element 55a, 55b can be thickened, oriented, or reinforced to resist deformation, especially when radially expanded. The reinforcement allows the structure of the expandable brush elements 55a, 55b to maintain its shape to scrape along the surface of the vessel to separate or scrape wall adherent clot material from the vessel wall. Debris from this scraping can be captured within an adjustable basket, as described above, or within other additional baskets such as debulking baskets.

As shown in FIGS. 12-18, the thrombectomy system 61 includes a delivery sheath 50 slidable over the brush element assembly 71. The delivery sheath 50 includes an elongated sheath body 52 having a proximal end 54 and a distal end 56. A sheath lumen 73 extends between the proximal end 54 and the distal end 56. The brush element assembly 71 is slidably received within the sheath lumen of the delivery sheath 50. The delivery sheath 50 is slidable axially over the brush element assembly 71 to selectively sheath and unsheathe the expandable brush elements 55a, 55b (or an adjustable basket, debulking baskets, etc., in some examples). The distal mouth 75 of the sheath body 52 is optionally reinforced to prevent distortion of the distal mouth as the distal end 56 is slid over the expandable brush elements 55a, 55b (or adjustable basket, debulking baskets, etc.). In at least one embodiment, the delivery sheath 50 includes a radiopaque band (such as a radiopaque band 58, as shown in FIG. 4) positioned proximate the distal end 56 for identifying the position of the distal end 56 with fluoroscopy or other imaging techniques during a surgical procedure.

In some embodiments, the expandable brush elements 55a, 55b are constructed with a piece of mesh (e.g., optionally a continuous woven mesh). An intermediate collar 65 is clamped (e.g., pinched, gathered, anchored, or crimped) at a middle portion of the expandable brush elements 55a, 55b. In these embodiments, the two expandable brush elements 55a, 55b of the mesh are radially expanded together. A proximal collar 67 clamps the proximal expandable brush element 55b with the outer catheter 12. In another example, a distal collar 63 clamps the distal expandable brush element 55a with the inner catheter 14. In this way, proximal movement of the inner catheter 14, for instance telescoping proximal movement relative to the outer catheter 12, causes the distal collar 67 to move toward the proximal collar 63 and the brush elements 55a, 55b, to radially expand. Conversely, the collars maintain the mesh portions proximate to the collars (and optionally underlying the collars) to remain compressed. Accordingly, the brush element assembly 71 has a corrugated or undulating profile, as shown in FIG. 14.

As shown in FIG. 12, the delivery sheath 50 includes a hub assembly 68 having a primary branch 70 and a side branch 72. The primary branch 70 is axially aligned with the elongated sheath body 52 and defines a lumen extending the lumen of the elongated sheath body 52. The primary branch 70 includes a locking element 74 such as a locking screw or a friction lock for engaging the outer catheter 12 of the brush element assembly 71. The locking element 74 can be tightened to fix the delivery sheath 50 at a particular position axially along the outer catheter 12. The side branch 72 defines a lumen fluidly connected to the lumen of the primary branch 70. The side branch 72 includes a valve 76 and a port 78. The port 78 can be used to deliver fluid for flushing the delivery sheath 50 or applying vacuum for aspirating the delivery sheath 50, while the valve 76 can control fluid flow through the side branch 72.

As shown in FIGS. 12 and 16, in an embodiment, the thrombectomy system 61 includes a handle assembly 80 operably connected to the proximal end of the brush element assembly 71. The handle assembly 80 includes a handle body 82 and a translation assembly 84 for axially advancing and withdrawing the inner catheter 14 of the brush element assembly 71. Generally, the outer catheter 12 is fixedly attached to the handle body 82 while the translation assembly 84 is configured to advance and withdraw the inner catheter 14 relative to the outer catheter 12 to contract and expand the expandable brush elements 55a, 55b, similar to the description of the handle assembly associated with FIG. 2-3 outlined above.

### VARIOUS NOTES AND ASPECTS

Aspect 1 can include subject matter (such as an apparatus, a method, a means for performing acts, or a machine readable medium including instructions that, when performed by the machine, that can cause the machine to perform acts), such as can include a thrombectomy system for mechanically engaging a clot, comprising: an inner catheter; an outer catheter defining an outer catheter lumen, wherein the inner catheter is slidably received within the outer catheter lumen such that the inner catheter is slidable relative to the outer catheter; an adjustable basket having a fenestrated structure extending between a proximal collar and a distal collar, wherein the proximal collar is attached to a distal end of the outer catheter and the distal collar is attached to a distal end of the inner catheter; at least one debulking element positioned on the outer catheter proximal to the adjustable basket; and wherein the fenestrated structure is radially expanded by sliding the inner catheter in a first direction relative to the outer catheter and is radially collapsed by sliding the inner catheter in a second direction opposite the first direction relative to the outer catheter.

Aspect 2 can include, or can optionally be combined with the subject matter of Aspect 1 to include, a handle assembly operably connected to the inner catheter to axially advance and withdraw the inner catheter and the adjustable basket.
can include, or can optionally be combined with the subject matter of Aspect 1 to include,

Aspect 3 can include, or can optionally be combined with the subject matter of Aspect 2 to include, wherein the first direction is a proximal direction and the fenestrated structure of the adjustable basket is radially expanded in response to the handle assembly axially withdrawing the inner catheter in the proximal direction.

Aspect 4 can include, or can optionally be combined with the subject matter of Aspects 2-3 to include, wherein the second direction is a distal direction and the fenestrated structure of the adjustable basket is radially contract in response to the handle assembly axially advancing the inner catheter in the distal direction.

Aspect 5 can include, or can optionally be combined with the subject matter of Aspects 1-4 to include, a procedural guidewire; wherein the inner catheter further defines an inner lumen for slidably receiving the procedural guidewire.

Aspect 6 can include, or can optionally be combined with the subject matter of Aspects 1-5 to include, wherein the inner catheter comprises: a tapered dilator tip positioned on the distal end of the inner catheter; wherein dilator tip tapers radially from a larger diameter proximal end of the dilator to a smaller diameter distal end of the dilator.

Aspect 7 can include, or can optionally be combined with the subject matter of Aspects 1-6 to include, wherein the fenestrated structure comprises: a body portion having a plurality of struts for engaging clot material and defining flow openings permitting fluid flow through the fenestrated structure; wherein the body portion defines a mouth at a proximal end of the body portion and a closed distal end tapering to the distal collar.

Aspect 8 can include, or can optionally be combined with the subject matter of Aspect 7 to include, wherein the fenestrated structure comprises: two legs extending from the proximal collar to the proximal end of the body portion; wherein the two legs are attached to the body portion proximate opposed sides of the mouth.

Aspect 9 can include, or can optionally be combined with the subject matter of Aspect 8 to include, wherein the mouth has at least two notched portions; wherein the at least two notched portions are each positioned between locations to which the two legs are attached to the body portion and opposite the other notched portion.

Aspect 10 can include, or can optionally be combined with the subject matter of Aspects 8-9 to include, wherein sliding the inner catheter in a proximal direction relative to the outer catheter enlarges at least the proximal end of body portion and an effective diameter of the mouth.

Aspect 11 can include, or can optionally be combined with the subject matter of Aspects 1-10 to include, wherein the at least one debulking element is at least one self-expanding debulking basket and comprises: a fenestrated structure extending between a proximal collar and a distal collar, the fenestrated structure defining a plurality of openings permitting fluid flow through the debulking element while capturing solid material within the self-expanding debulking basket; wherein the proximal collar is fixedly attached to the inner catheter and the distal collar is slidably attached to the inner catheter, wherein the distal collar slides distally along the inner catheter when the self-expanding debulking basket is radially constrained to collapse the self-expanding debulking basket and slides proximally along the inner catheter to expand the self-expanding debulking basket.

Aspect 12 can include, or can optionally be combined with the subject matter of Aspect 11 to include, wherein the fenestrated structure of the self-expanding debulking basket defines a mouth on a proximal end of the fenestrated structure.

Aspect 13 can include, or can optionally be combined with the subject matter of Aspects 1-12 to include, wherein the debulking element is spaced apart from the adjustable basket by a first predetermined distance.

Aspect 14 can include, or can optionally be combined with the subject matter of Aspects 1-13 to include, two debulking elements, wherein the two debulking elements are separated by a second predetermined distance.

Aspect 15 can include, or can optionally be combined with the subject matter of Aspects 1-14 to include, an expandable brush element positioned on the inner catheter distal to the adjustable basket; wherein the expandable brush element comprises a braided body having at least one radially projecting disc.

Aspect 16 can include, or can optionally be combined with the subject matter of Aspect 15 to include, wherein the expandable brush element comprises: a first radially projecting disc having a first outer diameter; and a second radially projecting disc having a second outer diameter; wherein the first outer diameter is different from the second outer diameter.

Aspect 17 can include, or can optionally be combined with the subject matter of Aspect 16 to include, wherein: the first outer diameter is about 12 to 14 mm and the second outer diameter is about 8 to 12 mm; and the adjustable basket is expandable to an outer diameter of about 16 mm.

Aspect 18 is a thrombectomy system for mechanically engaging clot, comprising: an inner catheter; an outer catheter defining an outer lumen, wherein the inner catheter is slidably received within the outer lumen such that the inner catheter is slidable relative to the outer catheter; an expandable brush element positioned between a proximal collar and a distal collar, wherein the proximal collar is attached to a distal end of the outer catheter and the distal collar is attached to a distal end of the inner catheter; and wherein the expandable brush element is radially expanded by sliding the inner catheter in a proximal direction relative to the outer catheter and is radially collapsed by sliding the inner catheter in a distal direction relative to the outer catheter.

Aspect 19 can include, or can optionally be combined with the subject matter of Aspect 18 to include, a handle assembly operably connected to the inner catheter to slide the inner catheter in the proximal direction and the distal direction.

Aspect 20 can include, or can optionally be combined with the subject matter of Aspects 18-19 to include, an adjustable basket having a fenestrated structure extending between an additional proximal collar and an additional distal collar; wherein: the additional proximal collar is attached to a distal end of the outer catheter and the additional distal collar is attached to a distal end of the inner catheter; and the expandable brush element is distal to the adjustable basket.

Aspect 21 can include, or can optionally be combined with the subject matter of Aspect 20 to include, wherein the fenestrated structure comprises: a body portion having a plurality of struts for engaging clot material and defining flow openings permitting fluid flow through the fenestrated structure; wherein the body portion defines a mouth at a proximal end of the body portion and a closed distal end tapering to the distal collar.

Aspect 22 can include, or can optionally be combined with the subject matter of Aspects 18-21 to include, a procedural guidewire; wherein the inner catheter further defines an inner lumen for slidably receiving the procedural guidewire.

Aspect 23 can include, or can optionally be combined with the subject matter of Aspects 18-22 to include, wherein the inner catheter comprises: a tapered dilator tip positioned on the distal end of the inner catheter; wherein the dilator tip tapers radially from a larger diameter proximal end of the dilator to a smaller diameter distal end of the dilator.

Aspect 24 can include, or can optionally be combined with the subject matter of Aspects 18-23 to include, wherein the expandable brush element comprises a braided body having at least one radially projecting disc.

Aspect 25 can include, or can optionally be combined with the subject matter of Aspects 18-24 to include, wherein the expandable brush element comprises: a first radially projecting disc having a first outer diameter; and a second radially projecting disc having a second outer diameter; wherein the first outer diameter is different from the second outer diameter.

Aspect 26 can include, or can optionally be combined with the subject matter of Aspect 25 to include, wherein: the first outer diameter is about 12 to 14 mm and the second outer diameter is about 8 to 12 mm; and the expandable brush element is expandable to an outer diameter of about 16 mm.

Aspect 27 can include, or can optionally be combined with the subject matter of Aspects 18-26 to include, at least one expandable debulking element positioned on the outer catheter proximal to the expandable brush element.

Aspect 28 can include, or can optionally be combined with the subject matter of Aspect 27 to include, wherein the expandable debulking element is spaced apart from the expandable brush element by a first predetermined distance.

Aspect 29 can include, or can optionally be combined with the subject matter of Aspect 28 to include, an additional expandable debulking elements, wherein the expandable debulking element and the additional expandable debulking element are separated by a second predetermined distance.

Aspect 30 is a method of removing a thrombus, comprising: delivering a thrombectomy system through a vessel to a thrombus, the thrombectomy system comprising: an inner catheter; an outer catheter defining an outer lumen, wherein the inner catheter is slidably received within the outer lumen; an adjustable basket having a fenestrated structure extending between a proximal collar and a distal collar, wherein the proximal collar is attached to a distal end of the outer catheter and the distal collar is attached to a distal end of the inner catheter; and at least one expandable debulking element positioned on the outer catheter proximal to the adjustable basket; crossing the thrombus with the thrombectomy system such that the adjustable basket is positioned distal to the thrombus; sliding the inner catheter in a proximal direction relative to the outer catheter to radially expand the adjustable catheter distal to the thrombus; deploying the expandable debulking element proximal to the expanded adjustable catheter; and pulling the thrombectomy system in a proximal direction toward a procedural sheath.

Aspect 31 can include, or can optionally be combined with the subject matter of Aspect 30 to include, wherein pulling the thrombectomy system in the proximal direction engages a distal portion of the thrombus with the adjustable basket.

Aspect 32 can include, or can optionally be combined with the subject matter of Aspect 31 to include, wherein the expandable debulking element is radially expanded within the thrombus to penetrate and engage clot material of the thrombus.

Aspect 33 can include, or can optionally be combined with the subject matter of Aspect 32 to include, wherein the expandable debulking element is spaced apart from the adjustable basket by a first predetermined distance.

Aspect 34 can include, or can optionally be combined with the subject matter of Aspects 31-33 to include, wherein: the expandable debulking element divides the thrombus; and pulling the thrombectomy system in the proximal direction includes: pulling a first portion of the thrombus with the expandable debulking element; and pulling a second portion of the thrombus with the adjustable basket.

Aspect 35 can include, or can optionally be combined with the subject matter of Aspects 31-34 to include, wherein the thrombectomy system comprises: two expandable debulking elements, wherein the two expandable debulking elements are separated by a second predetermined distance.

Aspect 36 can include, or can optionally be combined with the subject matter of Aspects 30-35 to include, wherein the thrombectomy system comprises: an expandable brush element positioned on the inner catheter distal to the adjustable basket.

Aspect 37 can include, or can optionally be combined with the subject matter of Aspect 36 to include, wherein the expandable brush element comprises a braided body having at least one radially projecting disc.

Aspect 38 can include, or can optionally be combined with the subject matter of Aspects 30-37 to include, moving the thrombectomy system in both a proximal and distal direction to scour a portion of the vessel.

Aspect 39 is a method of removing a thrombus, comprising: delivering a thrombectomy system through a vessel to a thrombus, the thrombectomy system comprising: an inner catheter; an outer catheter defining an outer lumen, wherein the inner catheter is slidably received within the outer lumen; an expandable brush element positioned between a proximal collar and a distal collar, wherein the proximal collar is attached to a distal end of the outer catheter and the distal collar is attached to a distal end of the inner catheter; crossing the thrombus with the thrombectomy system such that the expandable brush element is positioned distal to the thrombus; and sliding the inner catheter in a proximal direction relative to the outer catheter to radially expand the expandable brush element in close proximity to the thrombus.

Aspect 40 can include, or can optionally be combined with the subject matter of Aspect 39 to include, wherein the thrombectomy system further comprises an adjustable basket; and the method further comprises: unsheathing the adjustable basket proximal to the expandable brush element; and pulling the thrombectomy system in a proximal direction toward a procedural sheath.

Aspect 41 can include, or can optionally be combined with the subject matter of Aspect 40 to include, wherein the thrombectomy system comprises: at least one self-expandable debulking element positioned on the outer catheter proximal to the adjustable basket.

Aspect 42 can include, or can optionally be combined with the subject matter of Aspect 41 to include, wherein the adjustable basket has a fenestrated structure extending between an additional proximal collar and an additional distal collar, wherein the additional proximal collar is attached to a distal end of the outer catheter and the distal collar is attached to a distal end of the inner catheter.

Aspect 43 can include, or can optionally be combined with the subject matter of Aspects 40-42 to include, wherein the expandable brush element comprises a braided body having at least one radially projecting disc.

Aspect 44 can include, or can optionally be combined with the subject matter of Aspects 40-43 to include, moving the radially expanded expandable brush element in both a proximal and distal direction to scour a portion of the vessel.

Aspect 45 can include, or can optionally be combined with the subject matter of Aspect 44 to include, wherein the expandable brush element is spaced apart from the adjustable basket by a first predetermined distance.

Aspect 46 can include, or can optionally be combined with the subject matter of Aspect 45 to include, wherein the thrombectomy system comprises: two expandable debulking elements, wherein the two expandable debulking elements are separated by a second predetermined distance.

Aspect 47 is at least one machine-readable medium including instructions that, when executed by processing circuitry, cause the processing circuitry to perform operations to implement of any of Aspects 1-46.

Aspect 48 is an apparatus comprising means to implement any of Aspects 1-46.

Aspect 49 is a system to implement any of Aspects 1-46.

Aspect 50 is a method to implement any of Aspects 1-46.

Aspect 51 can include subject matter (such as an apparatus, a method, a means for performing acts, or a machine readable medium including instructions that, when performed by the machine, that can cause the machine to perform acts), such as can include a thrombectomy system for mechanically engaging a clot, comprising: an inner catheter; an outer catheter defining an outer catheter lumen, wherein the inner catheter is slidably received within the outer catheter lumen such that the inner catheter is slidable relative to the outer catheter; a thrombectomy element extending between a proximal collar and a distal collar, wherein the proximal collar is attached to a distal end of the outer catheter and the distal collar is attached to a distal end of the inner catheter; wherein the thrombectomy element is radially expanded by moving the inner catheter to decrease a distance between the distal collar and the proximal collar and is radially collapsed by moving the inner catheter to increase the distance between the distal collar and the proximal collar.

Aspect 52 can include, or can optionally be combined with the subject matter of Aspect 51 to include, wherein the thrombectomy element is an adjustable basket having a fenestrated structure extended between the proximal collar and the distal collar.

Aspect 53 can include, or can optionally be combined with the subject matter of Aspect 52 to include, include wherein the fenestrated structure is radially expanded by sliding the inner catheter in a first direction relative to the outer catheter and is radially collapsed by sliding the inner catheter in a second direction opposite the first direction relative to the outer catheter.

Aspect 54 can include, or can optionally be combined with the subject matter of Aspect 53 to include, a handle assembly operably connected to the inner catheter to axially advance and withdraw the inner catheter and the adjustable basket.

Aspect 55 can include, or can optionally be combined with the subject matter of Aspect 54 to include, wherein the first direction is a proximal direction and the fenestrated structure of the adjustable basket is radially expanded in response to the handle assembly axially withdrawing the inner catheter in the proximal direction.

Aspect 56 can include, or can optionally be combined with the subject matter of any one or more of Aspects 54-55 to include, wherein the second direction is a distal direction and the fenestrated structure of the adjustable basket is radially contract in response to the handle assembly axially advancing the inner catheter in the distal direction.

Aspect 57 can include, or can optionally be combined with the subject matter of any one or more of Aspects 52-56 to include, wherein the fenestrated structure comprises: a body portion having a plurality of struts for engaging clot material and defining flow openings permitting fluid flow through the fenestrated structure; wherein the body portion defines a mouth at a proximal end of the body portion and a closed distal end tapering to the distal collar.

Aspect 58 can include, or can optionally be combined with the subject matter of Aspect 57 to include, wherein the fenestrated structure comprises: two legs extending from the proximal collar to the proximal end of the body portion; wherein the two legs are attached to the body portion proximate opposed sides of the mouth.

Aspect 59 can include, or can optionally be combined with the subject matter of Aspect 58 to include, wherein the mouth has at least two notched portions; wherein the at least two notched portions are each positioned between locations to which the two legs are attached to the body portion and opposite the other notched portion.

Aspect 60 can include, or can optionally be combined with the subject matter of any one or more of Aspects 58-59 to include, wherein sliding the inner catheter in a proximal direction relative to the outer catheter enlarges at least the proximal end of body portion and an effective diameter of the mouth.

Aspect 61 can include, or can optionally be combined with the subject matter of any one or more of Aspects 51-60 optionally include wherein the thrombectomy element comprises: an adjustable basket having a fenestrated structure extended between the proximal collar and the distal collar; and an expandable brush element; wherein each of the adjustable basket and the expandable brush element is radially expanded by moving the inner catheter.

Aspect 62 can include, or can optionally be combined with the subject matter of Aspect 61 to include, wherein: the expandable brush element is positioned on the inner catheter distal to the adjustable basket; and wherein the expandable brush element comprises a braided body having at least one radially projecting disc.

Aspect 63 can include, or can optionally be combined with the subject matter of Aspect 62 to include, wherein the expandable brush element comprises: a first radially projecting disc having a first outer diameter; and a second radially projecting disc having a second outer diameter; wherein the first outer diameter is different from the second outer diameter.

Aspect 64 can include, or can optionally be combined with the subject matter of Aspect 63 to include, wherein: the first outer diameter is about 12 to 14 mm and the second outer diameter is about 8 to 12 mm; and the adjustable basket is expandable to an outer diameter of about 16 mm.

Aspect 65 is an apparatus comprising means to implement any of Aspects 51-64.

Aspect 66 is a system to implement any of Aspects 51-64.

Aspect 67 is a method to implement any of Aspects 51-64.

Each of these non-limiting aspects can stand on its own, or can be combined in various permutations or combinations with one or more of the other aspects.

The above description includes references to the accompanying drawings, which form a part of the detailed description. The drawings show, by way of illustration, specific embodiments in which the invention can be practiced. These embodiments are also referred to herein as “aspects” or “examples.” Such aspects or example can include elements in addition to those shown or described. However, the present inventors also contemplate aspects or examples in which only those elements shown or described are provided. Moreover, the present inventors also contemplate aspects or examples using any combination or permutation of those elements shown or described (or one or more features thereof), either with respect to a particular aspects or examples (or one or more features thereof), or with respect to other Aspects (or one or more features thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

The above description is intended to be illustrative, and not restrictive. For example, the above-described aspects or examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as aspects, examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed:

1. A thrombectomy system for mechanically engaging a clot, comprising:
an inner catheter;
an outer catheter defining an outer catheter lumen, wherein the inner catheter is slidably received within the outer catheter lumen such that the inner catheter is slidable relative to the outer catheter;
an adjustable basket having a fenestrated structure extending between a proximal collar and a distal collar, wherein the proximal collar is attached to a distal end of the outer catheter and the distal collar is attached to a distal end of the inner catheter;
at least one debulking element positioned on the outer catheter proximal to the adjustable basket; and
wherein the fenestrated structure is radially expanded by sliding the inner catheter in a first direction relative to the outer catheter and is radially collapsed by sliding the inner catheter in a second direction opposite the first direction relative to the outer catheter.

2. The thrombectomy system of claim 1, further comprising a handle assembly operably connected to the inner catheter to axially advance and withdraw the inner catheter and the adjustable basket.

3. The thrombectomy system of claim 2, wherein the first direction is a proximal direction and the fenestrated structure of the adjustable basket is radially expanded in response to the handle assembly axially withdrawing the inner catheter in the proximal direction.

4. The thrombectomy system of claim 2, wherein the second direction is a distal direction and the fenestrated structure of the adjustable basket is radially contract in response to the handle assembly axially advancing the inner catheter in the distal direction.

5. The thrombectomy system of claim 1, further comprising:
a procedural guidewire;
wherein the inner catheter further defines an inner lumen for slidably receiving the procedural guidewire.

6. The thrombectomy system of claim 1, wherein the inner catheter comprises:
a tapered dilator tip positioned on the distal end of the inner catheter;
wherein dilator tip tapers radially from a larger diameter proximal end of the dilator to a smaller diameter distal end of the dilator.

7. The thrombectomy system of claim 1, wherein the fenestrated structure comprises:
a body portion having a plurality of struts for engaging clot material and defining flow openings permitting fluid flow through the fenestrated structure;
wherein the body portion defines a mouth at a proximal end of the body portion and a closed distal end tapering to the distal collar.

8. The thrombectomy system of claim 7, wherein:
the fenestrated structure comprises two legs extending from the proximal collar to the proximal end of the body portion;
the two legs are attached to the body portion proximate opposed sides of the mouth;
the mouth has at least two notched portions;
the at least two notched portions are each positioned between locations to which the two legs are attached to the body portion and opposite the other notched portion; and
sliding the inner catheter in a proximal direction relative to the outer catheter enlarges at least the proximal end of body portion and an effective diameter of the mouth.

9. The thrombectomy system of claim 1, wherein the at least one debulking element is at least one self-expanding debulking basket and comprises:
a fenestrated structure extending between a proximal collar and a distal collar, the fenestrated structure defining a plurality of openings permitting fluid flow through the debulking element while capturing solid material within the self-expanding debulking basket;
wherein the proximal collar is fixedly attached to the inner catheter and the distal collar is slidably attached to the inner catheter, wherein the distal collar slides distally along the inner catheter when the self-expanding debulking basket is radially constrained to collapse the self-expanding debulking basket and slides proximally along the inner catheter to expand the self-expanding debulking basket.

10. The thrombectomy system of claim 9, wherein the fenestrated structure of the self-expanding debulking basket defines a mouth on a proximal end of the fenestrated structure.

11. The thrombectomy system of claim 1, wherein the debulking element is spaced apart from the adjustable basket by a first predetermined distance.

12. The thrombectomy system of claim 1, further comprising:
two debulking elements, wherein the two debulking elements are separated by a second predetermined distance.

13. A thrombectomy system for mechanically engaging clot, comprising:
an inner catheter;
an outer catheter defining an outer lumen, wherein the inner catheter is slidably received within the outer lumen such that the inner catheter is slidable relative to the outer catheter;
an expandable brush element positioned between a proximal collar and a distal collar, wherein the proximal collar is attached to a distal end of the outer catheter and the distal collar is attached to a distal end of the inner catheter; and
wherein the expandable brush element is radially expanded by sliding the inner catheter in a proximal direction relative to the outer catheter and is radially collapsed by sliding the inner catheter in a distal direction relative to the outer catheter.

14. The thrombectomy system of claim 13, further comprising a handle assembly operably connected to the inner catheter to slide the inner catheter in the proximal direction and the distal direction.

15. The thrombectomy system of claim 13, further comprising:
an adjustable basket having a fenestrated structure extending between an additional proximal collar and an additional distal collar;
wherein:
the additional proximal collar is attached to a distal end of the outer catheter and the additional distal collar is attached to a distal end of the inner catheter;
the expandable brush element is distal to the adjustable basket;
the fenestrated structure comprises a body portion having a plurality of struts for engaging clot material and defining flow openings permitting fluid flow through the fenestrated structure; and
the body portion defines a mouth at a proximal end of the body portion and a closed distal end tapering to the distal collar.

16. The thrombectomy system of claim 13, further comprising:
a procedural guidewire;
wherein the inner catheter further defines an inner lumen for slidably receiving the procedural guidewire.

17. The thrombectomy system of claim 13, wherein the inner catheter comprises:
a tapered dilator tip positioned on the distal end of the inner catheter;
wherein the dilator tip tapers radially from a larger diameter proximal end of the dilator to a smaller diameter distal end of the dilator.

18. The thrombectomy system of claim 13, wherein the expandable brush element comprises a braided body having at least one radially projecting disc.

19. The thrombectomy system of claim 13, wherein the expandable brush element comprises:
a first radially projecting disc having a first outer diameter; and
a second radially projecting disc having a second outer diameter;
wherein the first outer diameter is different from the second outer diameter.

20. The thrombectomy system of claim 19, wherein:
the first outer diameter is about 12 to 14 mm and the second outer diameter is about 8 to 12 mm; and
the expandable brush element is expandable to an outer diameter of about 16 mm.
